(19) United States
(12) Patent Application Publication
(10) Pub. No.: US 2017/0128480 A1
(43) Pub. Date: May 11, 2017

(54) Cardiac Glycosides for the Treatment of Hypercholesterolemia

(71) Applicants: Max A. Cayo, Milwaukee, WI (US); Stephen A. Duncan, Charleston, SC (US)

(72) Inventors: Max A. Cayo, Milwaukee, WI (US); Stephen A. Duncan, Charleston, SC (US)

(21) Appl. No.: 15/347,404

(22) Filed: Nov. 9, 2016

Related U.S. Application Data
(60) Provisional application No. 62/252,961, filed on Nov. 9, 2015.

(51) Int. Cl. A61K 31/7048; A61K 31/585

(52) U.S. Cl. CPC A61K 31/7048; A61K 31/585

(57) ABSTRACT

A method of treating hypercholesterolemia in a subject in need thereof comprising administering to the subject a therapeutically effective amount of a cardiac glycoside. In addition, a method of reducing, modulating or otherwise affecting production of ApoB-100-containing lipoproteins is also disclosed.

This application claims priority to and the benefit of application serial no. 62/252,961 filed Nov. 9, 2015—the entirety of which is incorporated herein by reference.

This invention was made with government support under ROI DK55743, UOI HG006398, F30 DK091994, DK087377, and P01-HL089471 awarded by the National Institutes of Health. The government has certain rights in the invention.

### BACKGROUND OF THE INVENTION

Cardiovascular disease, which primarily results from dysregulation of plasma lipoproteins (LDL, HDL), whose homeostasis are maintained by the liver, represents the largest singular cause of morbidity and mortality in human beings (Abegunde, D. O. et al., Lancet 370, 1929-1938 (2007)). Familial hypercholesterolemia (FH) is the most common inherited dyslipidemia, afflicting approximately 10 million people globally (Goldstein, J. L. & Brown, M. S., Annu Rev Genet 13, 259-289 (1979)).

FH is an autosomal-dominant inherited disorder caused primarily by mutations in the gene for the low-density lipoprotein receptor (LDLR) normally expressed on the surface of liver cells. FH individuals can be either heterozygous (HeFH) or homozygous (HoFH) for the FH gene mutation. Epidemiologic data indicates a HeFH prevalence of 1-in-500 and a HoFH prevalence of about 1-in-1 million in the general population. While optimal serum LDL-cholesterol (LDL-C) in humans is 100 mg/dl, patients with FH exhibit serum LDL-C levels ranging between 250-450 mg/dl in HeFH patients, and >500 mg/dl in HoFH patients. Depending on the severity, elevated LDL-C levels can lead to xanthomas, early onset arterial plaque formation, and severe and early onset coronary artery disease resulting in myocardial infarction and death (Rader, D. J. et al., J Clin Invest 111, 1795-1803 (2003)). The liver is critical in the pathogenesis of FH, evidenced by the fact that homozygous FH patients, with the most extreme elevations in serum LDL, are clinically cured by liver transplantation (Schmidt, H. H. et al., Clin Transplant 22, 180-184 (2008); and Kakaei, F. et al. Transplant Proc 41, 2939-2941 (2009)).

Hepatocytes are responsible for cholesterol synthesis and the secretion of lipoprotein particles necessary for transport of cholesterol throughout the body. Further, hepatocytes are involved in mechanisms for cholesterol clearance by conversion of cholesterol into bile acids. The current first-line and most effective clinical therapies for hypercholesterolemia in the general population are statin drugs (HMG-CoA reductase inhibitors), which act in a liver-specific context to block intracellular cholesterol synthesis leading to SREBP-mediated gene induction (including LDLR) and enhanced clearance of circulating LDL cholesterol. Although widely prescribed, complications associated with statin use are common, with 39% of individuals reporting complications in a randomized clinical trial. Most of the complications resulted in increased liver enzyme levels suggesting hepatocyte damage or myalgia. However memory loss and exercise induced acute pain are side effects associated with statin treatments. Although statins can be highly effective, there is a surprisingly wide variation of effectiveness between individuals, with >20% of patients showing a poor response to statin treatment.

Reflecting this lack of efficacious treatment options for FH patients, two new drugs, Lomitapide and Mipomersen, have recently received FDA approval for use in this patient group. Lomitapide inhibits the microsomal triglyceride transfer protein (MTTP), and Mipomersen is an anti-sense RNA oligo directed against ApoB-100 mRNA, a liver specific gene and the key protein component of VLDL and LDL particles. Both of these drugs reduce secretion of LDL-cholesterol independently of the LDLR receptor, rather than the LDLR-mediated enhancement of LDL clearance effected by statin drugs, and have been proven effective in the treatment of homozygous FH patients (Rader, D. J. & Kastelein, J. J. P., Circulation 129, 1022-1032 (2014)). However, both drugs have significant side effects, notably lipid accumulation in hepatocytes, and both are extraordinarily expensive and thus prohibitive for use in a large segment of patients.

### SUMMARY OF THE INVENTION

In light of the forgoing, it is an object of the present invention to provide a treatment for hypercholesterolemia, for both FH and genetically healthy, normal individuals, and related methods, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It can be an object of the present invention to provide an alternative treatment for hypercholesterolemia through an LDLR-independent mechanism.

It can be another object of the present invention to provide one or more methods of treating hypercholesterolemia for individuals that do not respond to or cannot tolerate statins.

It can be another object of this invention, alone or together with one or more of the preceding objectives, to provide a range of cardiac glycoside compounds for use in conjunction with a method for treating hypercholesterolemia, an indication thereof and/or a factor contributing thereto.

Other objectives, features, benefits and advantages of the present invention will be apparent from this summary and its descriptions of certain embodiments, and will be readily apparent to those skilled in the art having knowledge of hypercholesterolemia and associated treatment methods. Such objectives, features, benefits and advantages will be apparent from the above as taken into conjunction with the accompanying examples, data, figures and all reasonable inferences to be drawn therefrom.

In part, the present invention can be directed to a method for the treatment of hypercholesterolemia or familial hypercholesterolemia (FH) in a human subject in need thereof. Such a method can comprise administering to such a subject a therapeutically effective amount of a cardiac glycoside compound, for example, of a formula I:

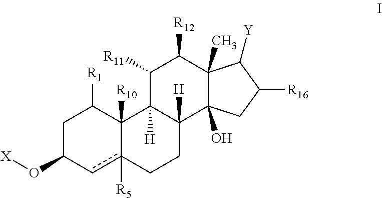

wherein X can be selected from hydrogen (H), monosaccharide, disaccharide and polysaccharide moieties; Y can be selected from 2H-pyran-2-one and 5H-furan-2-one moieties; R₁ can be selected from H, hydroxyl (—OH), monosaccharide, disaccharide and polysaccharide moieties; R₅ can be selected from H, methyl and —OH moieties; R₁₀ can be selected from H, methyl, hydroxymethyl, —OH and formyl (—(H)C═O) moieties; R₁₁ and R₁₂ can be independently selected from H and —OH moieties; R₁₆ can be selected from H, —OH and acetate (—OC(═O)CH₃) moieties; and [symbol] can represent either a single bond or a double bond; or R₁ and R₅ can be an oxy (or —O—), and together with the X—O oxy group, wherein X is absent, form a fused heterocyclic ring containing the R₁, R₅ and X—O oxy groups.

In certain embodiments, such a compound can be a cardenolide compound, wherein Y can be such a 5H-furan-2-one-4-yl moiety. In certain other embodiments, such a compound can be a bufadienolide compound, wherein Y can be such a 2H-pyran-2-one-5-yl moiety.

In part, the present invention can also be directed to a method of reducing, modulating or otherwise affecting production of ApoB-100-containing lipoproteins. Such a method can comprise providing a compound of the sort discussed above or illustrated elsewhere herein; and contacting a cellular medium comprising a hepatocyte producing and/or expressing an ApoB-100-containing lipoprotein with such a compound in an amount effective to reduce, modulate or otherwise affect hepatocyte production of ApoB-100. Such a method can thereby reduce serum LDL-cholesterol levels.

Alternatively, in part, the present invention can also be directed to a method of inhibiting, modulating or otherwise affecting a sodium/potassium-ATPase pump mechanism of a human hepatocyte. Such a method can comprise providing a compound of the sort discussed above or illustrated elsewhere herein; and contacting a cellular medium comprising a hepatocyte comprising a sodium/potassium-ATPase pump mechanism with such a compound in an amount effective to inhibit, modulate or otherwise affect pump activity. Such a method can thereby affect hepatocyte ApoB-100 production.

Alternatively, in part, the present invention can provide a method for treating a human subject with a liver that produces LDL and/or VLDL particles of a size and number. Such a method can comprise administering to the subject a therapeutically effective amount of a cardiac glycoside compound of the sort discussed above or illustrated elsewhere herein such that the compound can lower the VLDL/LDL/ApoB particle number and/or increase the size thereof, in the subject.

### BRIEF DESCRIPTION OF THE DRAWINGS

FIGS. 1A-B. (A) is a graph revealing that an ELISA efficiently and reliably (z′=0.88) distinguish between cells that do (iPSC-derived hepatocytes, top) and do not (iPSC-derived endoderm, bottom) secrete ApoB-100; (B) shows that ELISA also distinguishes between the levels of ApoB-100 in the medium of iPSC-derived hepatocytes treated with either vehicle (DMSO, blue) or the protein synthesis inhibitor cyclohexamide (CHX, less than 100%).

FIGS. 2A-B. (A) is a graph showing the post-drug:pre-drug ratios ApoB-100 identified in the culture medium from the primary screen, wherein circles represent individual drugs within the library, and box-and-whisker plots summarize the mean and distributions for each plate (30 plates); (B) is a graph showing z-scores (lines=±3) of the effect of 2320 drugs (spheres) on the concentration ApoB-100 in the culture medium of treated cells.

FIG. 3 is a box and whisker plot demonstrating that 13 small molecules, including 5 cardiac glycosides that are identified as hits in the primary screen, plus 4 additional cardiac glycosides present in the library, reproducibly (n=3, p≦0.05) inhibit the levels of ApoB-100 measured in the culture medium compared to cells treated with vehicle (DMSO, dashed line).

FIG. 4 is sequence of graphs showing dose response curves generated from the treatment of HepG2 cells with various cardiac glycosides at 1, 5, 20, 78, 312, 1250, and 5000 nM.

FIGS. 5A-C provide bar graphs showing the percent of lipidated ApoB-100 secreted into the medium over 24 hours after the treatment of primary human hepatocytes with cardiac glycosides at 0, 20, 80, 310, and 1,250 nM. The level of ApoB-100 in untreated cells is set to 100% and error bars reflect the s.e.m, n=3.

FIG. 6 shows a time-course experiment indicating that cardiac glycosides (50 nM) reduce the rate of secretion of ApoB-100 from patient-specific iPSC-derived hepatocytes at the earliest time points tested, as compared to DMSO (upper plot). Re-uptake of newly secreted lipoproteins is blocked by incubating the cells with heparin throughout the duration of the experiment.

FIG. 7A-B. (A) provides bar graphs showing that an antibody recognizing lipidated human ApoB-100 (LDL, VLDL) specifically detects ApoB-100 by ELISA in the serum of humanized but not un-transplanted FRGN mice; (B) is a graph showing that the level of human ApoB-100 closely correlates with the level of human albumin in the sera of humanized mice over time.

FIGS. 8A-C. (A) is a graph showing the ratio of human albumin to human ApoB-100 found in the serum of avatar mice harboring hepatocytes from two different donors (donor A circles, donor B squares); (B) is a graph showing the percent change in the concentration of ApoB-100 found in the serum of avatar mice (donor A circles, donor B squares) treated with vehicle DMSO, Digoxin, or Proscillaridin. The graph shows that treatment of avatar mice (donor A circles, donor B squares) with vehicle DMSO, Digoxin, or Proscillaridin have no effect on albumin levels; (C) is a graph showing the relative change in human ApoB-100 normalized to the change in human albumin following treatment with DMSO (left) cardiac glycosides Digoxin (middle) and Proscillaridin (right).

FIG. 9 provides graphs depicting the serum concentrations of human ApoB-100 and human albumin in individual avatar mice before and after treatment with (DMSO) vehicle, Digoxin, or Proscillaridin over a 48-hour period.

FIG. 10 is a graph showing concentration of LDL in the serum of a cohort of patients (grey circles) when on or off treatment with either an angiotensin-converting-enzyme inhibitor (ACE-i), a statin, or a cardiac glycoside. The bar shows the mean LDL concentration.

FIG. 11 is a graph showing the concentration of albumin in the serum of patients (grey circles) when on or off treatment with the indicated drugs. The bar shows the mean albumin concentration.

FIGS. 12A-B is a graph showing serum albumin (A) and LDL (B) measurements in the same patients before and after treatment with cardiac glycosides. The bar shows the mean concentration and the grey lines indicate the direction of change in each patient when off (left diamonds) or on (right diamonds) the medication indicated (Statin=control, Digoxin=experimental group).

FIGS. 13A-B (A) is a graph of the results of a FPLC showing the impact of Digoxin and Proscillaridin on the increase in the size of LDL/VLDL particles and the increase of HDL particle number in serum (FIG. 13B, enhanced).

### DETAILED DESCRIPTION OF CERTAIN EMBODIMENTS

The invention relates to a method for the treatment of familial hypercholesterolemia (FH) in a human subject in need thereof comprising administering to the subject a therapeutically effective amount of a cardiac glycoside compound, for example, of a formula I:

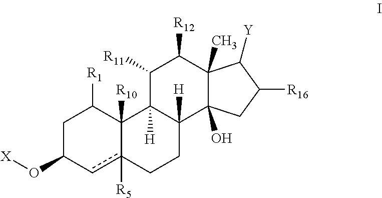

wherein X is selected from a group consisting of H, a monosaccharide, a disaccharide and a polysaccharide; Y is selected from a group consisting of 2H-pyran-2-one and furan-2(5H)-one; R₁ is selected from a group consisting of H, —OH, a monosaccharide, a disaccharide and a polysaccharide; R₅ is selected from a group consisting of H, methyl and —OH; R₁₀ is selected from a group consisting of H, methyl, hydroxymethyl, —OH and —(H)C═O; R₁₁ and R₁₂ are independently selected from a group consisting of H and —OH; R₁₆ is selected from a group consisting of H, —OH and —OC(═O)CH₃); and [symbol] represents either a single bond or a double bond; or R₁ and R₅ are an oxy (or —O—), and together with the X—O oxy group, wherein X is absent, form a fused heterocyclic ring containing the R₁, R₅ and X—O oxy groups.

As used herein, “cardiac glycoside” or “cardiac glycoside compound” is an organic compound that act on the contractile force of the cardiac muscle. Typically, the cardiac glycoside includes a steroid portion, a glycoside (or saccharide) portion (usually bonded to the C-3 of the steridl portion), and a lactone portion (usually a furan-2(5H)-one or a 2H-pyran-2-one bonded to the C-17 of the sterol). The genus of a cardiac glycoside is shown in formula II. A preferred sub-genus of formula II is formula I depicted above.

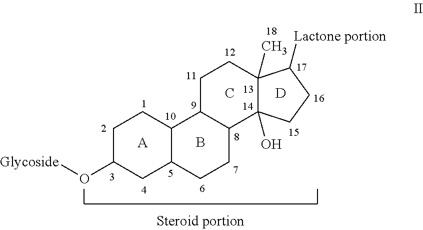

In formula II, the steroid portion is represented by four fused rings, ring A, ring B, ring C and ring D, and by oxy at C-3, the hydroxyl at C-14 and the methyl (or —CH₃) at C-13, the carbon of the C-13 methyl being C-18. As discussed above, the lactone portion is either a 2H-pyran-2-one or 5H-furan-2-one moiety, as depicted below.

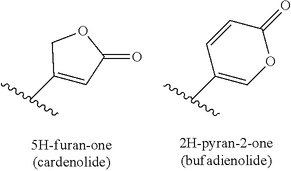 [SMILES: *C1=CC(=O)OC1 ; *c1ccc(=O)oc1]

By “fused ring” is meant two or more ringed structures that are bonded to each other at one or more adjacent atoms. For example, the compounds of formula I and formula II are fused at adjacent carbon atoms between ring A and ring B, ring B and ring C, and ring C and ring D. Ring B and ring C are fused to both ring A and ring C and ring B and ring D, respectively. The result is a four-membered fused ring system. By “fused heterocyclic ring” is meant a two or more fused ring system containing one or more heteroatoms, i.e. atoms other than carbon and preferably oxygen, nitrogen or sulfur. Such a fused heterocyclic ring is formed, for example, between C-1, C-3 and C-5, wherein each of C-1, C-3 and C-5 is directly attached to an oxygen (example depicted below).

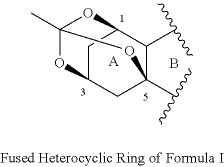

The compounds of formula I and formula II also embrace “stereochemical isomers”. The term “stereochemical isomer” as used herein, refers to isomers that differ from each other only in the way the atoms are oriented in space. The two stereoisomers particularly of importance in the instant invention are “enantiomers” and “diastereomers” depending on whether or not the two isomers are mirror images of each other (enantiomers). Within the molecule (of formula I and formula II), a “stereocenter” (or chiral center) is an atom bearing groups such that an interchanging of any two groups leads to a stereoisomer. Put another way, a chiral center is a tetrahedral atom (usually carbons) that has four different substituents. Each chiral center in a molecule will be either “R” or “S”. Thus, the “ [symbol] ” (or wedge) and “ [symbol] ” (or dashed) found in formula I represents two bonds that are drawn in the plane of the page, the former bond is drawn going out of the page (and towards the viewer, or wedged), and latter bond is drawn going behind the page (and away from the viewer, or dashed).

While certain non-limiting compounds are represented herein as having one or more stereocenters, more generally, various compounds useful in conjunction with the methods of this invention are without stereochemical or configurational limitation. Accordingly, any stereocenter can be (S) or (R) with respect to any other stereocenter(s). Further, it will be understood by those skilled in the art that any one or more of the compounds relating to this invention can be provided as part of a pharmaceutical composition comprising a pharmaceutically-acceptable carrier component for use in conjunction with a treatment method or medicament.

Specific examples of cardiac glycoside compounds according to the invention include, but are not limited to, Digoxin, Convallatoxin, Proscillaridin, Digitoxin, Lanatoside C, Ouabain (Strophanthin), Gitoxin, Peruboside, Strophanthidin, Digoxigenin, and the like. Such compounds and various other cardiac glycosides in accordance with this invention are commercially-available from sources well-known to those skilled in the art.

### In Vitro Study

Apolipoprotein B100 (ApoB-100) is a protein that plays a role in moving cholesterol around the body. It is a form of low-density lipoprotein (LDL). Mutations in the ApoB-100 gene can cause familial hypercholesterolemia. The ApoB-100 gene is a liver specific gene and the key protein component of very low-density lipoprotein (VLDL) and low-density lipoprotein (LDL) particles.

In an embodiment, cardiac glycoside compounds are screened from a drug library and are found to reduce ApoB-100 production lipoprotein. Preferably, a method for reducing, modulating or otherwise affecting production of ApoB-100-containing lipoproteins is provided, the method comprising providing a cardiac glycoside compound of the sort discussed above or illustrated elsewhere herein; and contacting a cellular medium comprising a hepatocyte producing an ApoB-100-containing lipoprotein with the cardiac glycoside compound in an amount effective to reduce, modulate or otherwise affect hepatocyte production of ApoB-100, thereby reducing serum LDL-cholesterol levels.

FH patient-specific, induced pluripotent stem cells (iPSC or PSC) are efficiently differentiated to hepatocytes. These iPSC-derived hepatocytes fail to traffic exogenous LDL to endosomes and are unable to increase LDL clearance in response to statin treatment. Moreover, compared to controls, the patient-specific iPSC-derived hepatocytes possess elevated secretion of lipidated ApoB-100 (Cayo, M. A., et al., Hepatology 56, 2163-2171 (2012), incorporated herein by reference).

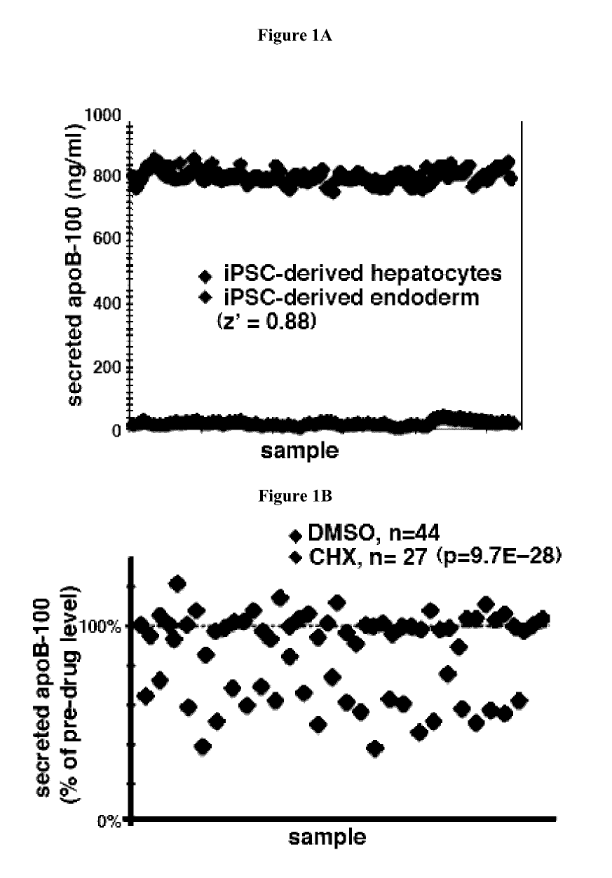

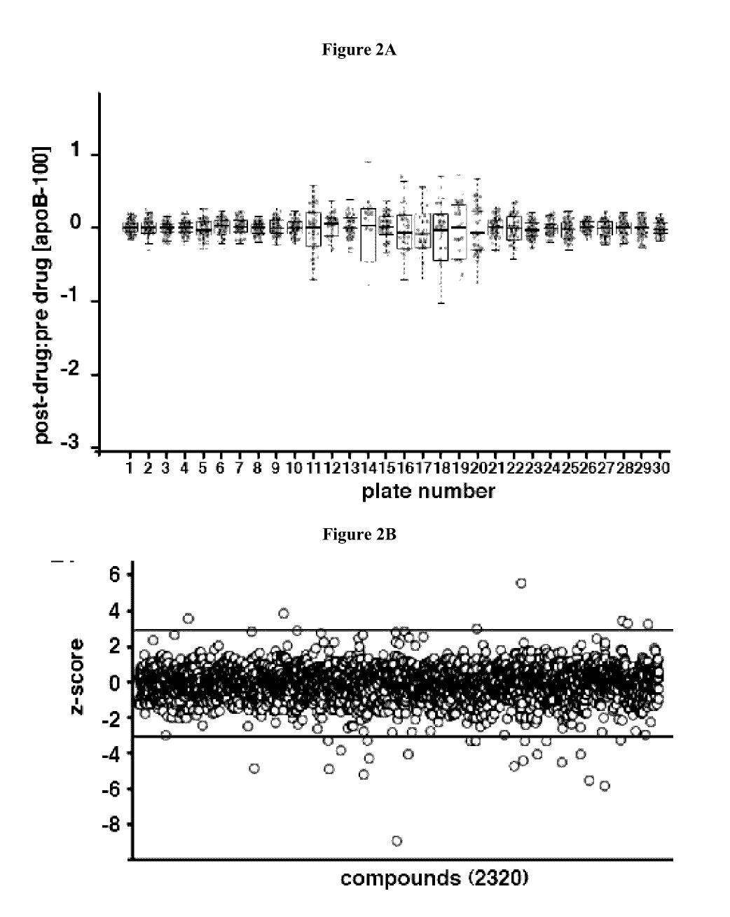

As a result, the observed increase in ApoB-100 secretion by FH patient-specific iPSC-derived hepatocytes is theorized to represent a screenable phenotype for identification of novel LDL-lowering drugs that are effective in FH patients and therefore act in an LDLR-independent fashion. ApoB-100 secretion is measured by enzyme linked immunosorbent assay (ELISA) using an antibody that specifically detects lipidated apoB-100, which represents lipoprotein particles secreted by the iPSC-derived hepatocytes. A comparison of the ELISA performance on positive and negative control cells (an analysis referred to as a Z-factor), taking into account the magnitude difference (effect size) and spread (standard deviation) between and among the positive and negative control samples, indicates that this assay is excellent for application to screening platforms (Z-factor=0.88) (FIG. 1). In this context, the ELISA is able to readily distinguish the effect of treating iPSC-derived hepatocytes with vehicle or the protein synthesis inhibitor cyclohexamide (p=9.7×10⁻²⁸) (FIG. 1). FH patient-specific iPSC cells are differentiated to hepatocyte-like cells in 96-well plates and the levels of lipidated ApoB-100 are measured before and after the application of 2320 small molecules from the SPECTRUM collection drug library (FIGS. 2A-B).

Using the before-drug and after-drug ApoB-100 concentration for each individual well (see the Examples below; each well represents 1 drug), a post:pre drug ratio of ApoB-100 is generated (referred to as delta-apoB-100; FIGS. 2A and 2B) and primary hits are identified by z-score analysis (FIG. 2B) as described by Zhang (Zhang, X. D., J Biomol Screen 16, 775-785 (2011)). A z-score is calculated for each compound, representing a multiple of the standard deviation of all the compounds included in the parent plate (30 plates total—FIG. 2A). Compounds with a z-score of ≦−3 (decreased apoB-100) or ≧3 (increased apoB-100) are considered primary hits as designated by applying the “3-sigma” rule (see Zhang). Satisfying these criteria are 8 drugs that increased and 21 drugs that reduced secreted ApoB-100. ApoB-100 ELISAs are then performed using all primary hit compounds in triplicate and the impact of each drug is compared to treatment with vehicle (DMSO) controls.

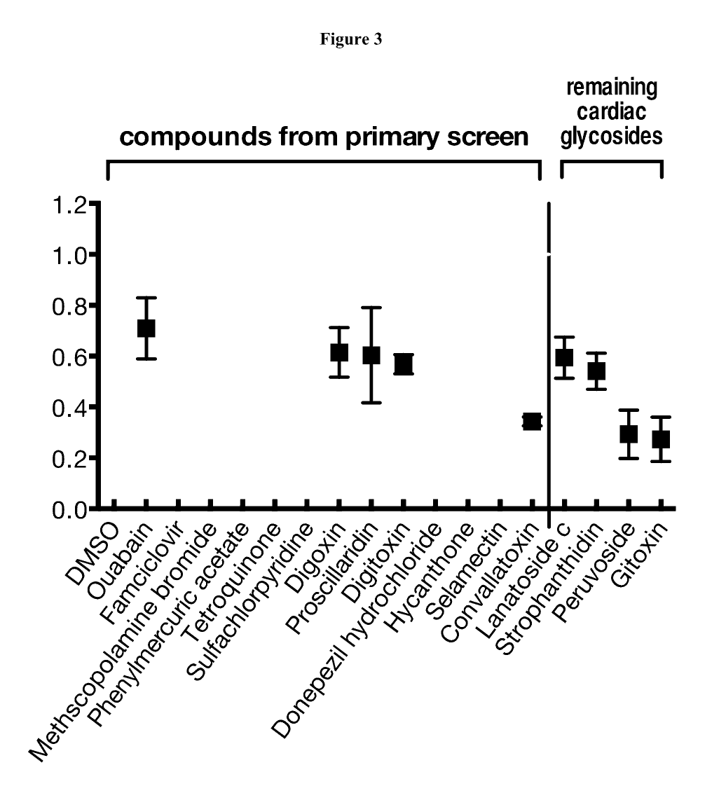

Of the 29 primary hits, 55% are found to be reproducible (p≦0.05, t-test) leaving 13 compounds which reduce the level of secreted ApoB-100 (FIG. 3). Of the compounds that reproducibly decreased secretion, 5 are cardiac glycosides. If the stringency of the z-score is reduced to ≦−2.0 in the primary screen, 7 of 9 total cardiac glycosides present in the SPECTRUM library satisfy the criterion. Subsequently, all 9 of the cardiac glycosides are tested in triplicate in the ApoB-100 ELISA assay to determine whether they shared property of reducing secretion of ApoB-100 (FIG. 3). Remarkably, every cardiac glycoside tested reduces ApoB-100 levels compared to pre-treatment levels and to DMSO controls (p≦0.05). Reductions range from 71% (Ouabain) to 27% (Gitoxin) of DMSO controls (FIG. 3). The names and structures of various cardiac glycosides, including the 9 identified in the screen, are provided in Table 1 below. Table 1 also provides the structure of cholesterol for comparison.

TABLE 1

| Drug/ | |
|---|---|
| Compound | Chemical Structure |
| Cholesterol | 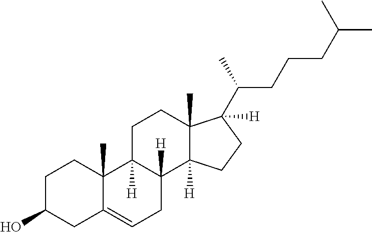 |
| Digoxin | 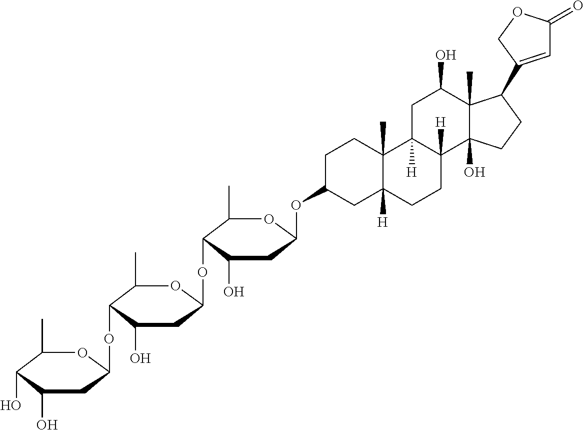 |
| Convallatoxin | 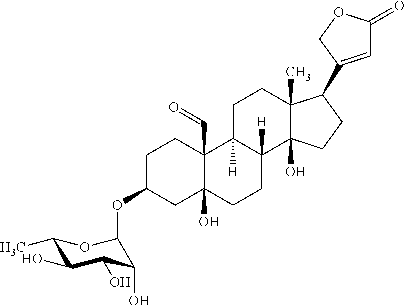 |
| Proscillaridin | 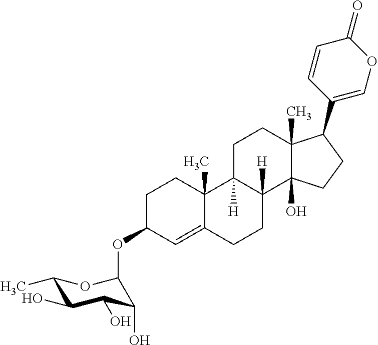 |
| Digitoxin | 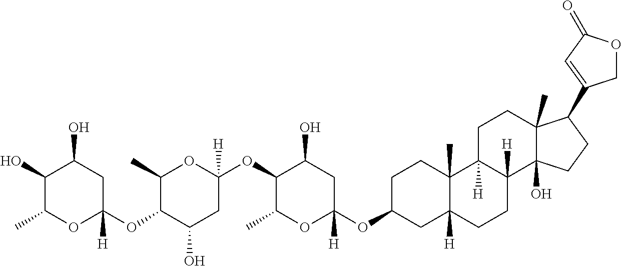 |
| Lanatoside C | 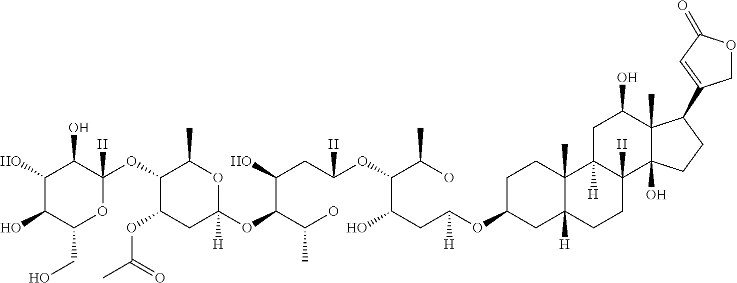 |
| Ouabain (Strophanthin) | 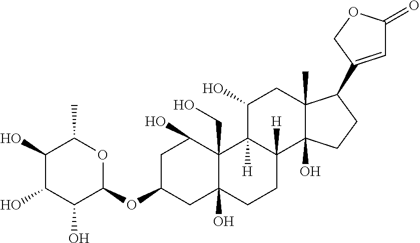 |
| Gitoxin | 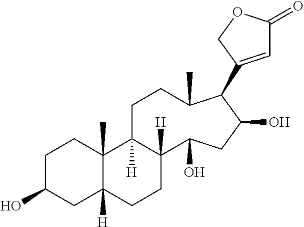 |
| Peruvoside | 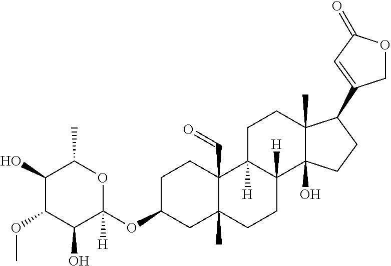 |
| Strophanthidin | 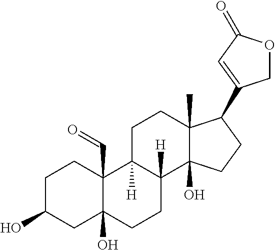 |
| Digoxigenin | 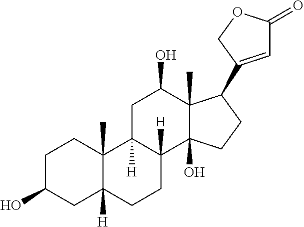 |

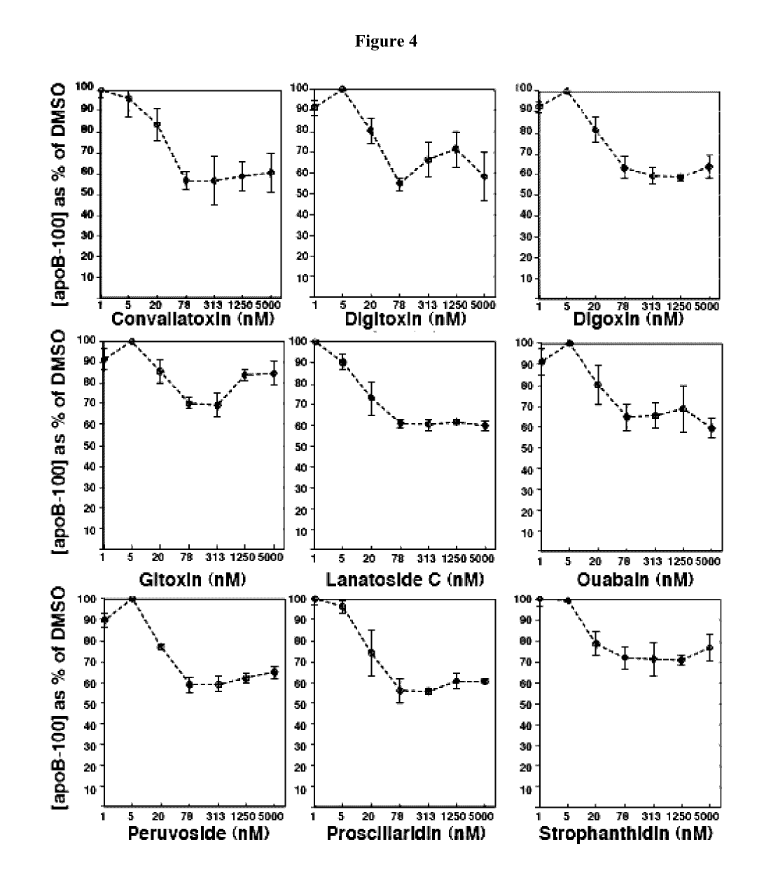

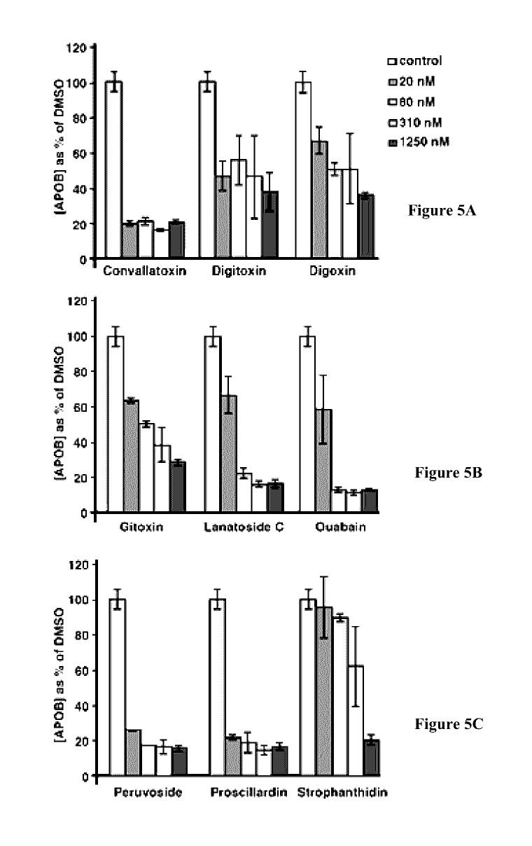

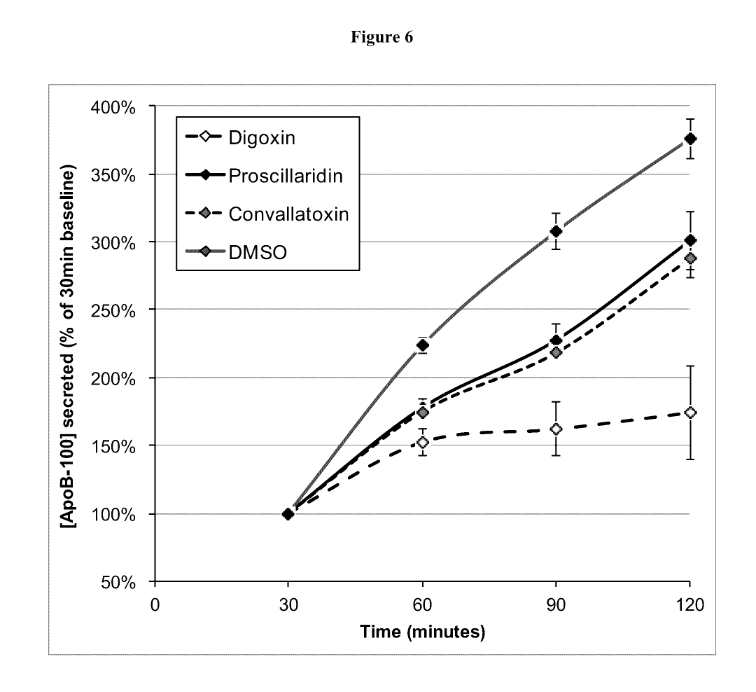

It was thought that the ability of cardiac glycosides to lower ApoB-100 should not be restricted to hoFH cells. To determine whether the cardiac glycosides reduce ApoB-100 levels in cells possessing a WT LDLR, dose-response curves are generated using both HepG2 hepatoma cells (FIG. 4) and plated primary human hepatocytes (FIGS. 6A-C). Each of the 9 cardiac glycosides tested reduces ApoB-100 production with an EC50≦20 nM in these experiments (FIG. 5 and FIGS. 6A-C). Based on these data, it is concluded that cardiac glycosides have an unappreciated, previously unreported, ability to lower the levels of secreted ApoB-100 from both LDLR-deficient and wild-type hepatocytes in vitro.

Furthermore, when tested at 50 nM concentrations in a time-course experiment using FH patient-specific iPS-derived hepatocytes, cardiac glycosides reduced the media concentration of ApoB-100 at the earliest time points, within 30 minutes to 1 hour, indicating that the governing pathway, which is LDLR-independent owing to the lack of functional LDLR in the patient background, is likely secretion of ApoB-100 rather than re-uptake. The reason is two-fold. First, reuptake is not significant at the very low concentrations of ApoB-100 present in culture at these earliest time points, and second because this effect is seen despite blocking re-uptake using heparin incubation through the assay (FIG. 6). Heparin blocks re-uptake of newly secreted lipoproteins by physically wrapping around the particles and preventing their interaction with receptors (Mahley, R. W., Weisgraber, K. H. & Innerarity, T. L., Biochimica et Biophysica Acta (BBA)-Lipids and Lipid Metabolism 575, 81-91 (1979), incorporated herein by reference).

LDL-Cholesterol Levels in Human Patients Treated with Cardiac Glycosides

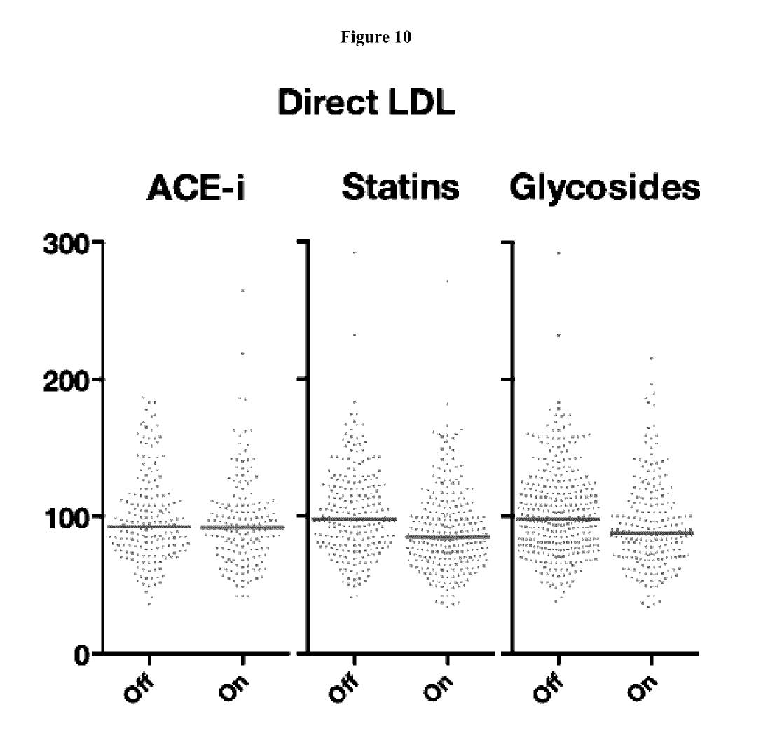

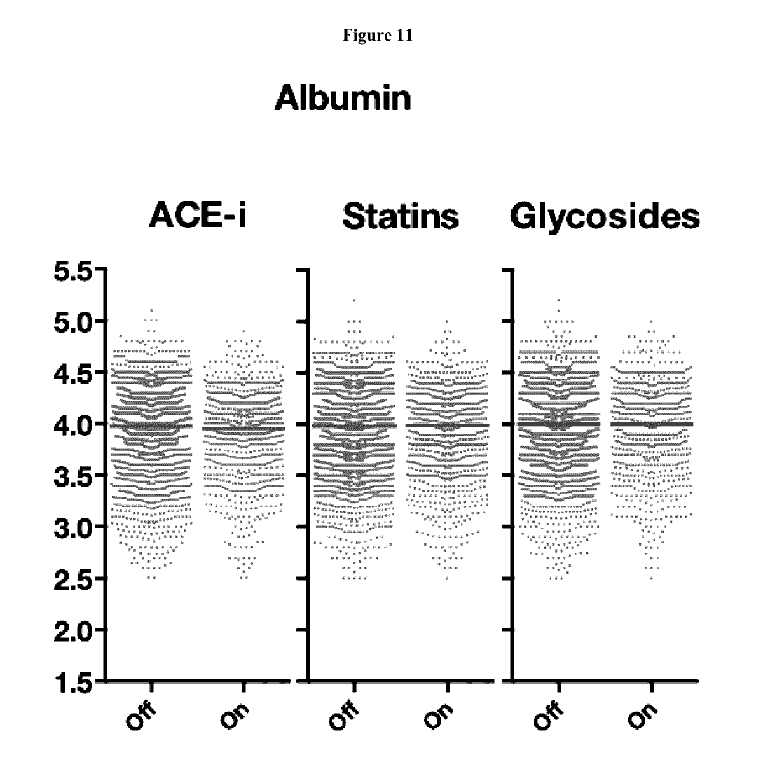

To determine whether cardiac glycosides have LDL-cholesterol lowering properties in humans, medical records are examined, in a retrospective and de-identified manner, of patients that entered the Froedtert Hospital/Medical College of Wisconsin clinics using next-generation database query tools and bioinformatics. A cohort of patients are identified that have, at any time, been treated with a cardiac glycoside within this hospital system (n=5,493), and who possess at least one direct LDL-cholesterol laboratory result (n=645). In a parallel and identical process, subsets of patients are identified within this cohort that had been treated with an angiotensin-converting-enzyme (ACE) inhibitor (n=380), which has no documented effect on LDL-cholesterol levels, or a statin (n=507), which serves as a positive control owing to these drugs' well-documented effect on serum LDL concentrations. Since the time-frame during which a patient is taking a specific medication is known, it is possible to retrieve serum LDL-cholesterol and serum albumin measurements recorded when patients were either on or off of drug treatment (FIG. 10 and FIG. 11). Laboratory test results (Direct LDL-C, albumin) are flagged as either on-drug or off-drug using the start and end dates of the medication orders for each patient. Following this hypothesis, treatment with an ACE-inhibitor, stratified by on-versus off-drug, is not associated with any difference in serum concentration of either LDL-cholesterol (p=0.441) or albumin (p=0.2388) (FIG. 10 and FIG. 11). Further, treatment with a statin is associated with a reduced mean serum LDL-cholesterol concentration: 103.6±2.252 mg/dL in off-drug patients versus 89.79±1.935 mg/dL (p<0.0001) for on-drug patients, while albumin levels are unaffected in these analyses (FIG. 10 and FIG. 11). When the same analysis is applied to treatment with cardiac glycosides, a reduced mean serum LDL-cholesterol of 103.1±1.827 mg/dL in the on-drug patients to 93.99±2.280 mg/dL off-drug (p=0.0019) is revealed, with no effect on serum albumin (FIG. 10 and FIG. 11). Therefore, the magnitude of the reduction associated with cardiac glycoside treatment is very near to the reduction seen in the statin analysis.

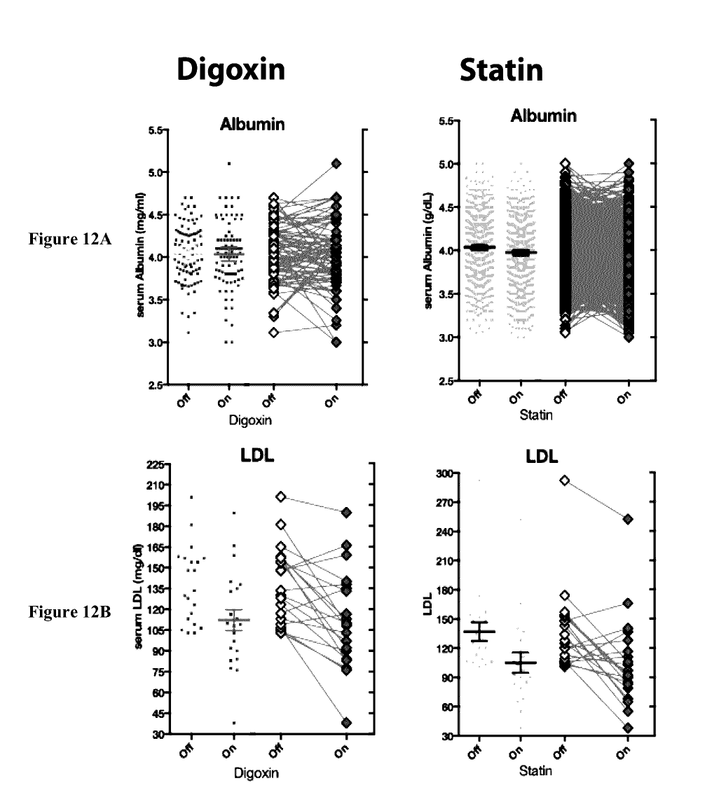

In conceptualizing the available patient data within the de-identified EMR database, it is recognized that a more definitive human analysis is achieved by comparing LDL-cholesterol measurements in a paired approach, using LDL measurements in an individual patient, before and during treatment with a given drug. Beginning once more with the database of all patient records, patients are identified with measurements of serum albumin (n=91) or LDL-cholesterol (n=21) taken both before and after treatment with either a cardiac glycoside (or a statin, which again is used a positive control (FIGS. 12A-B). The serum albumin concentrations in these patients is once more unaffected by drug treatment (−0.06 and +0.02 g/dL, respectively). In contrast, patients display a mean serum LDL-cholesterol reduction of 31.8±8.6 mg/dL (p=0.0016) during treatment with statins and 25.6±6.3 mg/dL (p=0.0006) with cardiac glycosides. Of the 21 patients examined in the cardiac glycoside treated cohort, LDL-cholesterol levels in 16 dropped substantially following the administration of a cardiac glycoside.

### In Vivo Study—Effect of Cardiac Glycosides on Serum Cholesterol Levels in Humanized Avatar Mice

Although the foregoing retrospective analyses of electronic medical records were convincing, it was understood that in contrast to a clinical trial, interpretation of results could be confounded by random variables such as diet, adherence, other drug use, physical condition and the like. To test whether the cardiac glycosides identified in the primary screening using HF patient-specific iPSC-derived hepatocytes and validated in vitro cause similar effects in vivo, mouse models are used. Unfortunately, the control of cholesterol homeostasis differs dramatically between mice and humans. Mice have lipid profiles predominated by HDL, while humans possess a majority of the more atherogenic LDL. Additionally, virtually all ApoB-containing lipoproteins produced by the livers of mice contain the shorter apoB-48, rather than full-length ApoB-100, which makes up nearly 100% of that secreted by the livers of humans.

To circumvent this, avatar mice are generated in which human hepatocytes, following transplantation into the FAH-null mouse model and cycling of the drug 2-(2-nitro-4-trifluoromethylbenzoyl)-1,3-cyclohexanedione (NTBC, or Nitisinone), are able to replace and repopulate the endogenous murine liver cells, eventually comprising nearly 100% of the liver parenchyma in these animals (Azuma, H. et al., Nat Biotechnol 25, 903-910 (2007), incorporated herein by reference). Such mice harboring humanized livers have previously shown in the literature to adopt a typical human lipoprotein profile. Such animals, therefore, provide an ideal model in which to test the cholesterol-reducing properties of cardiac glycosides on human hepatocytes in vivo.

Human albumin, used to track the extent of “humanization” of these animals' livers, eventually reaches approximately 10 mg/mL in the serum. Lending rationale to this approach, FAH-null mice harboring humanized livers have recently been shown to adopt a human-like lipoprotein profile. Such animals therefore provide an ideal model in which to test the LDL-cholesterol-lowering properties of cardiac glycosides. Primary hepatocytes are transplanted from two donors, 1) donor A—a 53-year old female and 2) donor B—a 17-year-old male.

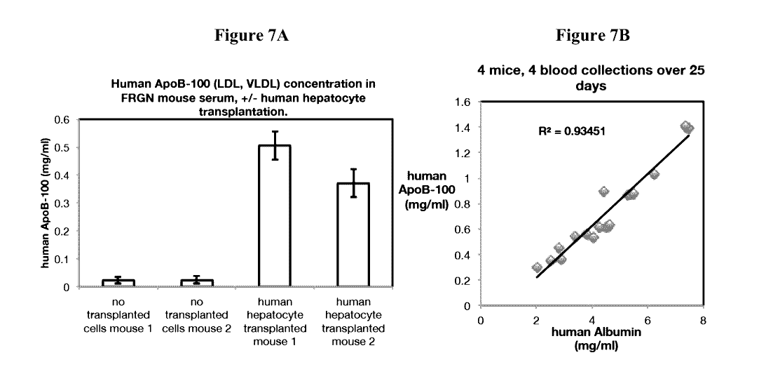

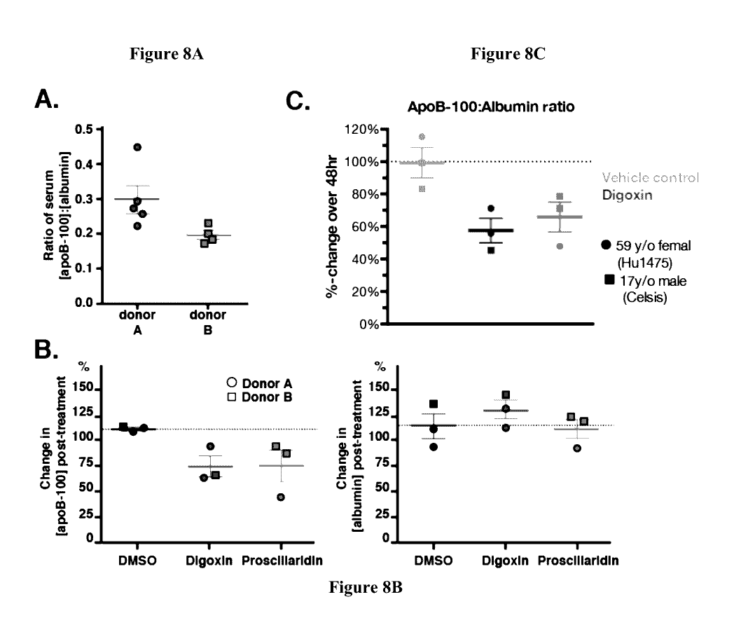

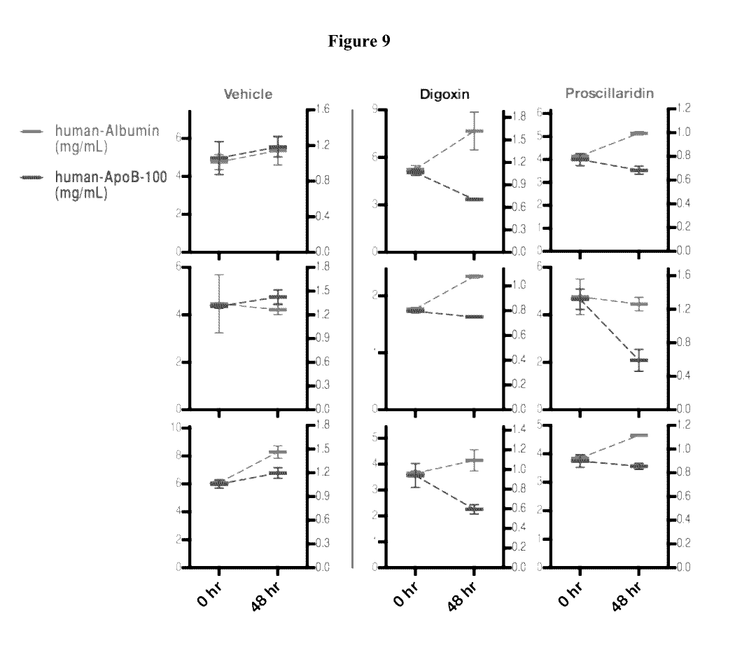

Prior to experimentation using cardiac glycosides in these animals, measuring serum ApoB-100 and albumin levels by ELISA is confirmed using human-specific antibodies that do not return any measureable concentration in FAH-null mice without transplanted human hepatocytes (FIG. 7A), as well as that these two concentrations correlate with one another linearly (FIG. 7B). Prior to any drug or vehicle treatment, the ratio of ApoB-100 to albumin is approximately 30% higher in the serum of avatars repopulated with hepatocytes from donor A compared to donor B (p=0.032) (FIG. 8A). Human ApoB-100 and albumin is then measured before and after treatment with DMSO (vehicle), Digoxin or Proscillaridin, over a 48-hour period of time (FIG. 8C). In contrast to the vehicle control treatment, which had no effect on serum human ApoB-100 or albumin concentration, treatment of the avatar mice with digoxin or proscillaridin significantly reduces serum ApoB-100 levels (p≦0.05) (FIG. 8B and FIG. 9, lower plots). The concentration of human albumin is unaffected in the serum of the same animals (FIG. 8B).

### Na+/K+-ATPase in Hepatocytes

In another embodiment, a method of inhibiting, modulating or otherwise affecting a sodium/potassium-ATPase pump mechanism of a human hepatocyte is provided, the method comprising providing a compound of the sort discussed above or illustrated elsewhere herein; and contacting a cellular medium comprising a hepatocyte comprising a sodium/potassium-ATPase pump mechanism with such a compound in an amount effective to inhibit, modulate or otherwise affect pump activity, thereby affecting hepatocyte ApoB-100 production.

In the prior art, cardiac glycosides typically inhibit Na⁺/K⁺-ATPase activity in cardiac myocytes in the micromolar range (See, e.g., Werdan et al., 1984, Biochem Pharmacol, 33, 55-70.) In contrast thereto and demonstrated herein, ApoB-100 production by hepatocytes is inhibited at nanomolar concentrations of such compounds, with the direct implication that the therapeutic dose used to treat hypercholesterolemia by targeting the liver can be significantly reduced compared to that used to treat heart failure thereby increasing efficacy and minimalizing risk.

In particular, the Na+/K+-ATPase is made up of 3 subunits: α, β, and γ (FIG. 15). The primary tissue-specific diversity of the channel derives from the α and β subunits, each of which has 3 isoforms. The ouabain binding site, also the binding site of other cardiac glycoside compounds, resides within the α subunit between the 1^{st} and 2^{nd} transmembrane domains and is extracellular. In liver cells, the α1 and β1/β2 subunit isoforms are expressed. The central nervous system and pigmented cells of the retina possess the widest array of α and β Na+/K+-ATPase subunits, and overall the α1β1 subunit combination is the most prevalent and widely distributed in all tissues (Blanco, G., Semin Nephrol 25, 292-303 (2005), incorporated herein by reference).

Differential expression of these isoforms of the subunits is regulated not only tissue to tissue but during development, in response to endocrine signals, and in disease states in various tissues, signaling it's critical role in each of these processes. Na+/K+-ATPase is inhibited by ouabain and other cardiac glycosides, but there are endogenous mechanisms that regulate the function of the enzyme, namely, the quantity of the pump present in the membrane of cells, which is regulated at the level of synthesis & degradation of the subunits of the pump as well as redistribution and recycling of the proteins within intracellular stores (Ewart, H. S. & Klip, Am J Physiol 269, C295-C311 (1995); and Therien, A. G. & Blostein, R., Am J Physiol Cell Physiol 279, C541-C566 (2000), both incorporated herein by reference. Insulin stimulation causes redistribution of Na+/K+-ATPase to the cell surface in muscle cells (Féraille, E. et al., Mol Biol Cell 10, 2847-2859 (1999), incorporated herein by reference). Dopamine causes endocytosis of Na+/K+-ATPase in central neurons, which effect is blocked by glutamate (Sottej eau, Y. et al., Biochemistry 49, 3602-3610 (2010), incorporated herein by reference). Generally, the a subunit is responsible for the heterogeneity in ouabain responsiveness, and the β subunit is responsible for modulating the interaction of Na+ and K+ ions with the pump (Segall, L., Daly, S. E. & Blostein, R., J Biol Chem 276, 31535-31541 (2001), incorporated herein by reference).

The ouabain sensitivity of the a subunits has been determined to be approximately 40 and 80 nM for the α2, and α3 subunits, and about 100-fold higher, in the uM range for the α1 subunit, which is somewhat higher, in the most commonly expressed α1 subunit, than the dosage range that results in reduced apoB-100 secretion by hepatocytes in the experiments within this dissertation (Kolansky, D. M. et al., FEBS Lett 303, 147-153 (1992), incorporated herein by reference). The sensitivity has been studied the most heavily in the human heart, which expresses all three a subunits, where the results are more controversial because multiple ouabain binding sites have been reported (Erdmann, E., Werdan, K. & Brown, L., Eur Heart J5 Suppl F, 297-302 (1984), incorporated herein by reference). Furthermore, endogenous concentrations of K+ have been shown to modulate the ouabain sensitivity, leading to even more heterogeneity. One proposed function accomplished by this range of ouabain sensitivities of the various Na+/K+-ATPase isozymes in different tissues under different conditions is the fact that there are endogenous ouabain-like compounds produced by the body and present in many of these tissues, suggesting the presence of endogenous mechanisms for regulating function within the tissues via Na+/K+-ATPase (Blaustein, M. P., Am J Physiol 264, C1367-C1387 (1993), incorporated herein by reference).

### Cardiac Glycosides Increase the Size of LDL/VLDL Particles

In a further embodiment, a method for treating a human subject with a liver that produces small LDL and VLDL particles is provided, the method comprising administering to the subject a therapeutically effective amount of a cardiac glycoside compound of the sort discussed above or illustrated elsewhere herein such that the compound lowers the VLDL/LDL/ApoB particle number, by increasing the size thereof, in the subject.

The method is particularly important because a newer view of atherosclerosis and heart disease is that particle size and number rather than total LDL-cholesterol is the important factor. Smaller LDL particles are more atherogenic, therefore, the fewer larger particles are preferred. Mouse data suggests that this effect occurs with cardiac glycoside treatment.

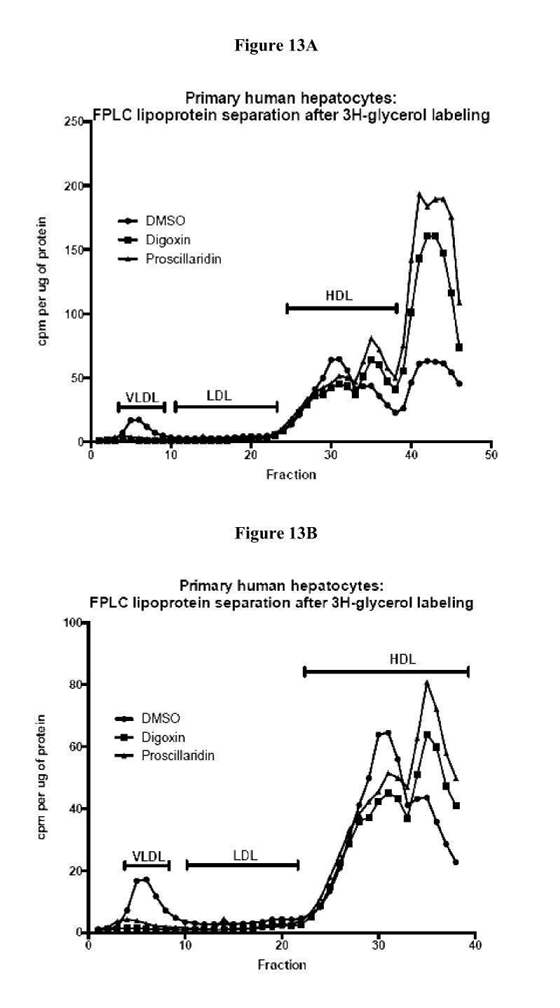

The in vitro data shows that VLDL particle size is increased, while HDL particle number is also increased. The experiment is a simple collection of cell culture media and then uses a known assay to measure VLDL/LDL/HDL by HPLC. FIGS. 13A-B show that HDL goes up with treatment, and that there is more cholesterol per LDL particle, indicating that the particles are more dense.

Methods of the present invention can also, as would be understood by those skilled in the art, be extended to or include methods using or in conjunction with a pharmaceutical composition comprising a cardiac glycoside compound of the sort described herein in a physiologically or otherwise suitable formulation. In a some embodiments, the present invention includes one or more cardiac glycoside compounds, of the sort forth above, formulated into compositions together with one or more physiologically tolerable or acceptable diluents, carriers, adjuvants or vehicles that are collectively referred to herein as carriers. Compositions suitable for such contact or administration can comprise physiologically acceptable sterile aqueous or nonaqueous solutions, dispersions, suspensions or emulsions. The resulting compositions can be, in conjunction with the various methods described herein, for administration or contact with a cellular medium comprising a hepatocyte, a sodium/potassium-ATPase pump mechanism and/or ApoB-100 expressed or otherwise present therein. Whether or not in conjunction with a pharmaceutical composition, “contacting” means that a hepatocyte and one or more cardiac glycoside compounds are brought together for purpose of binding and/or complexing such a compound to the hepatocyte and/or an ATPase enzyme. Amounts of a compound effective to inhibit hepatocyte or enzyme activity may be determined empirically, and making such determinations is within the skill in the art. Modulation, inhibition or otherwise affecting hepatocyte enzyme activity includes both reduction and/or mitigation, as well as elimination of enzyme activity and/or ApoB-100 and/or LDL-cholesterol production.

Regarding compositions useful in conjunction with methods of this invention, preparation of pharmaceutical formulations or compositions include the step of bringing the active ingredient(s) into association with a carrier and, optionally, one or more accessory ingredients. In general, the formulations are prepared by uniformly and intimately bringing the active ingredient(s) into association with liquid carriers, or finely divided solid carriers, or both, and then, if necessary, shaping the product. For example, standard pharmaceutical formulation techniques can be employed, such as those described in Remington's Pharmaceutical Sciences, Mack Publishing Company, Easton, Pa.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredient(s). The active ingredient(s) may also be administered as a bolus, electuary or paste.

In solid dosage forms of the invention for oral administration (capsules, tablets, pills, dragees, powders, granules and the like), the prodrug(s), active ingredient(s) (in their micronized form) is/are mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethyl-cellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient(s) moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient(s) therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient(s) can also be in microencapsulated form.

Liquid dosage forms for oral administration of the active ingredient(s) include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethylacetate, butyl alcohol, benzyl benzoate, propylene glycol, glycol, oils (in particular, cottonseed, groundnut, corn, germ, olive, castor and sesame oils), glycerol, amyl alcohol, tetrahydrofuryl polyethylene glycols and fatty acid esters of sorbitan, and mixtures thereof.

Besides inert diluents the oral compositions can also include adjuvants such as wetting agents, emulsifying and suspending agents, sweetening, flavoring, coloring, perfuming and preservative agents. Suspensions, in addition to the active ingredient(s), may contain suspending agents as, for example, ethoxylated alcohols, polyoxyethylene sorbitol and sorbitan esters, microcrystalline cellulose, aluminum metahydroxide, bentonite, agar-agar and tragacanth, and mixtures thereof.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Another mode of delivery for the compounds of the present invention may be delivery via the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethyl aspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

Pharmaceutical compositions of this invention suitable for parenteral administration comprise the active ingredient(s) in combination with one or more pharmaceutically-acceptable sterile isotonic aqueous or nonaqueous solutions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

In some cases, in order to prolong the effect of the active ingredient(s), it is desirable to slow the absorption of the drug from subcutaneous or intramuscular injection. This may be accomplished by the use of a liquid suspension of crystalline or amorphous material having poor water solubility. The rate of absorption of the active ingredient(s) then depends upon its/their rate of dissolution which, in turn, may depend upon crystal size and crystalline form. Alternatively, delayed absorption of parenterally-administered active ingredient(s) is accomplished by dissolving or suspending the active ingredient(s) in an oil vehicle. Injectable depot forms are made by forming microencapsule matrices of the active ingredient(s) in biodegradable polymers such as polylactide-polyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions which are compatible with body tissue. The injectable materials can be sterilized for example, by filtration through a bacterial-retaining filter.

Preferably the composition delivered in the form of an injectable dosage form comprise a biocompatible polymer, a compatible form of the presently disclosed compounds and a biocompatible solvent which solubilizes the biocompatible polymer wherein the weight percents of the biocompatible polymer, the instant and biocompatible solvent are based on the total weight of the complete composition.

It is understood by those skilled in the art that dosage amount will vary with the activity of a particular cardiac glycoside compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically-acceptable salt thereof, or related composition may be administered in two or more sub-doses, administered separately over an appropriate period of time.

Regardless of composition or formulation, those skilled in the art will recognize various avenues for medicament administration, together with corresponding factors and parameters to be considered in rendering such a medicament suitable for administration. Accordingly, with respect to one or more non-limiting embodiments, the present invention provides for use of one or more cardiac glycoside compounds for the manufacture of a medicament for therapeutic use in the treatment of various disease states relating to hypercholesterolemia.

### Examples of the Invention

The following non-limiting examples and data illustrate various aspects and features relating to the methods of the present invention, including the treatment of hypercholesterolemia using cardiac glycoside compounds. In comparison with the prior art, the present methods provide results and data which are surprising, unexpected and contrary thereto. While the utility of this invention is illustrated through the use of several glycoside compounds and related compositions which can be used therewith, it will be understood by those skilled in the art that comparable results are obtainable with various other compounds and/or compositions, as are commensurate with the scope of this invention.

### Example 1—Generation of Induced Pluripotent Stem Cells(iPSCs)

Generation of iPSCs from a hoFH patient is as described in the literature. (See, Cayo et al., 2012, Hepatology, 56, 2163-71.) A patient was previously shown to have a deletion in exon 17 of the maternal allele which was a null mutation, and an A-G transition in exon 17 of the paternal allele, which encodes a receptor that is unable to internalize LDL. (See, Davis et al., 1986, Cell, 45, 15-24.) Hepatocyte-like cells generated from these hoFH iPSCs faithfully recapitulated the pathophysiology associated with liver of hoFH patients. (Cayo et al., supra.) More specifically, hoFH hepatocytes failed to traffic exogenous LDL to endosomes and were unable to increase LDL clearance in response to statin treatment. Moreover, compared to controls, the hoFH iPSC-derived hepatocytes had elevated levels of APOB in the culture medium. (Cayo et al., supra.)

### Example 2—High-Throughput Screening Using JD iPS-Derived Hepatocytes

Pre-drug and post-drug apoB-100 concentrations are determined for each compound in the SPECTRUM library using a standard curve and four-parameter logistic (4PL) regression model. The pre-drug and post-drug apoB-100 concentrations are combined and expressed as a delta-apoB-100 ratio (post-drug [apoB-100]:pre-drug [apoB-100]), and a Z-score is generated for each individual compound using the delta-apoB-100 ratio with the standard deviation of the delta-apoB-100 ratio from the parent drug plate (30 drug plates total). Primary hits are validated in secondary replicate experiments (n=3), and statistical significance is determined by a Student's t-test.

### Example 3—Drug Library

The SPECTRUM collection drug library is purchased from Microsource Discovery Systems INC. The library consists of 2320 small molecules (http://www.msdiscovery.com/spectrum.html) and has been previously used to identify drugs for repurposing (Weisman, J. L. et al., Chem Biol Drug Des 67, 409-416 (2006); Kocisko, D. A. et al., J Virol 77, 10288-10294 (2003); Fagan, R. L. et al., PLoS One 8, e78752 (2013); and Wang, C. et al., Eur Urol 58, 418-426 (2010), all of which are incorporated herein by reference). This library contains approximately 1,000 compounds that have reached clinical trials in the United States, as well as approximately 250 drugs that are approved for human use in Europe/Asia. The library also contains approximately 800 compounds that are termed natural products, drug-like compounds that have predicted biological activity.

### Example 4—Enzyme Linked Immunosorbent Assay (ELISA)

A sandwich ELISA to detect human albumin in tissue culture supernatants and mouse sera uses a 1:100 dilution of a capture human albumin coating antibody (Bethyl laboratories, A80-129A) and a 1:85,000 dilution of a Horseradish Peroxidase (HRP) conjugated human albumin detection antibody (Bethyl laboratories, A80-129P). Bound antibody is detected using 3,3′,5,5′-tetramethylbenzidine (TMB) and the concentration of albumin in each sample is determined by comparing to a standard curve (Bethyl laboratories, RS10-110). Human lipidated apoB-100 is detected using a commercial sandwich ELISA (product code: 3715-1H-6; MabTech, Inc.) and detected using TMB. The concentration of apoB-100 is determined by comparing to a standard curve using lipidated apoB-100 supplied by the manufacturer.

### Example 5—Humanized FRGN Mice

Fah^{-/-}Rag2^{-/-}IL2 gr^{-/-}NOD (FRGN) mice are generated and supplied by Dr. Markus Grompe. Female FRGN breeders are kept healthy by transplanting with C57b1/6J bone marrow. All FRGN breeder mice are provided with drinking water supplemented with 8 mg/1NTBC. FRGN mice used for transplant are maintained by supplementing drinking water with 1 mg/l NTBC. To generate avatars, 1×10⁶ human primary hepatocytes are introduced into 6-8 week old FRGN mice by splenic injection. NTBC is withdrawn from the drinking water and mice are left for 7-days. Mice are then transferred to 8 mg/l NTBC for 3-days. The mice are cycled 7-days off drug followed by 3-days on drug for 2 months. After 2 months of cycling, the mice are kept without NTBC for around 15 days or until they lose 15% of body weight at which point they are returned to NTBC for 4-days. This cycle is maintained for the life of the animal. The extent of engraftment is measured by determining the human serum albumin and human apoB-100 levels by ELISA, as describe in Azuma, H. et al. cited above, and Bissig, K. -D. et al. and Ellis, E. C. S. et al. (Bissig, K. -D. et al., J Clin Invest 120, 924-930 (2010); and Ellis, E. C. S. et al., PLoS One 8, e78550 (2013), both incorporated herein by reference). Human hepatocytes for transplantation are obtained either from Thermo Fisher/Life Technologies (donor A, Hu1475) or from Celsis In Vitro Technologies, INC (donor B). Donor A is a deceased 53-year old, Caucasian female whose cause of death is unknown (occasionally used alcohol (wine, 2 glasses daily) and an ex-smoker (1 ppd×30 years, stopped in 2007)). Donor B is a deceased 17-year old Caucasian who died from head trauma due to a motor vehicle accident (occasionally used alcohol and dipping tobacco).

### Example 6—Drug Treatment of Humanized FRGN Mice

For drug treatment studies, highly repopulated FRGN mice (>1 mg/ml serum human albumin) are maintained using 0.15 mg/L NTBC drinking water prior to and during 48-hour drug treatment experiments. Blood samples are collected using 4 mm Goldenrod animal lancets (Medipoint, Inc.) and BD Microtainer EDTA-coated plasma collection tubes (Becton Dickinson Vacutainer Systems). Serum samples are collected at times 0, 24, and 48 hours and at the identical time of day. The mice are weighed daily and treated with either 0.5 mg/kg cardiac glycoside (Digoxin), 0.6 mg/kg/day (Proscillaridin) or vehicle control (5% DMSO in sterile saline) by i.p. injection at time points 0, 16, and 40 hours. (The dose selected was ⅛ of the reported LD₅₀ for each drug and within the published range 0.1 mg-2 mg/kg/day.) Serum concentrations of human albumin and human apoB-100 are determined using ELISA. Statistical significance is determined using Student's t-tests.

### Example 7—Bioinformatic Analysis of Human Patient Medical Records

Research subjects: The Froedtert/MCW Hospital and Clinics Epic Systems electronic medical record is queried using the MCW i2b2 Clinical & Translational Research Informatics Data Warehouse (CTRI-CRDW) and Cohort Discovery Tool. De-identified medical records are extracted from patients whose charts included ≧1 medication order for a cardiac glycoside, and ≧1 Direct LDL-C laboratory result.

Research objectives: Laboratory test results (Direct LDL-C, albumin) are flagged as either on-drug or off-drug using the start and end dates of the medication orders for each patient. If a patient's records contains multiple laboratory result values within a single on-drug or off-drug time window, the multiple values are combined into an average, such that each data point plotted or analyzed further represents a unique patient's average laboratory value for the indicated time window.

Sample size: 5,493 patients.

Data inclusion/exclusion criteria: For Direct LDL-C, any laboratory test results with values below 30 mg/dL are excluded (3 of 1,192 total), along with 4 results that are flagged as “ERROR” within the patients' charts. For albumin, any tests flagged with “ERROR” are removed, as are pathological results <3 or >5.4 g/dL (4,333 of total 59,532 lab results).

Statistical analysis: Statistical significance is determined using Student's t-tests. Paired data for individual human patients (on-drug versus off-drug) is analyzed using a paired t-test. Individual statistical tests are indicated in the body of the article and the figure legends.

### Example 8—Cardiac Glycosides Increase the Size of LDL/VLDL Particles

Cell culture media (the serum samples of example 5) is collected and then using a known assay (see, Example 3) VLDL/LDL/HDL is measured by FPLC HPLC. The experiment was performed by Evanthia Pashos Ph.D, a post-doctoral researcher at the University of Pennsylvania. Dr. Pashos was instructed what concentration of drugs to use, how long to incubate, and to record lipoprotein profiles for HDL, LDL, VLDL. The results are depicted in FIG. 16A and FIG. 16B, showing that treatment with Digoxin and Proscillaridin (310 nm) increase HDL secretion, reduce VLD/LDL total quantity and increase VLD/LDL particle size. Hepatocyte secretion was collected and analyzed over a 4-hr collection period.

As demonstrated, hepatocyte-like cells can be used in a high throughput screen to identify existing drugs to treat hypercholesterolemia. In addition to HoFH patients, such drugs can, therefore, be useful for reducing LDL-cholesterol in patients that do not respond to or cannot tolerate statins or other therapeutics of the prior art.

We claim:

1. A method of using a cardiac glycoside compound to modulate production of Apolipoprotein B100 (ApoB-100), said method comprising:
providing a cardiac glycoside compound of a formula 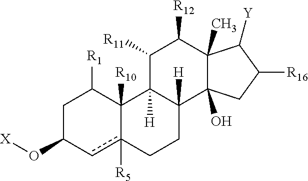
wherein X is selected from hydrogen (H), monosaccharide, disaccharide and polysaccharide moieties; Y is selected from 2H-pyran-2-one and 5H-furan-2(5H)-one moieties; R₁ is selected from H, hydroxyl (—OH), monosaccharide, disaccharide and polysaccharide moieties; R₁₀ is selected from H, methyl, hydroxymethyl, —OH and formyl (—(H)C═O) moieties; R₁₁ and R₁₂ are independently selected from H and —OH; R₁₆ is selected from H, —OH and acetate (—OC(═O)CH₃) moieties; and [symbol] represents either a single bond or a double bond, providing R₅ is selected from H, methyl and —OH moieties where said bond is a single bond.

2. The method of claim 1 wherein said compound is selected from bufadienolide compounds comprising a 2H-pyran-2-one-5-yl moiety and cardenolide compounds comprising a 5H-furan-2-one-4-yl moiety.

3. The method of claim 2 wherein said compound is selected from digoxin, convallatoxin, proscillaridin, digitoxin, lanatoside C, ouabain, gitoxin, peruboside, strophanthidin and digoxigenin.

4. The method of claim 3 wherein said compound is provided is provided in a composition comprising a nanomolar concentration thereof.

5. The method of claim 1 wherein said cellular medium is in a mammalian subject.

6. A method of reducing LDL-cholesterol levels, said method comprising: providing a cardiac glycoside compound of a formula 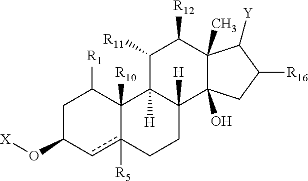
wherein X is selected from hydrogen (H), monosaccharide, disaccharide and polysaccharide moieties; Y is selected from 2H-pyran-2-one and 5H-furan-2(5H)-one moieties; R₁ is selected from H, hydroxyl (—OH), monosaccharide, disaccharide and polysaccharide moieties; R₁₀ is selected from H, methyl, hydroxymethyl, —OH and formyl (—(H)C═O) moieties; R₁₁ and R₁₂ are independently selected from H and —OH; R₁₆ is selected from H, —OH and acetate (—OC(═O)CH₃) moieties; and [symbol] represents either a single bond or a double bond, providing R₅ is selected from H, methyl and —OH moieties where said bond is a single bond; and
administering said compound to a mammalian subject expressing ApoB-100, said compound in an amount sufficient to reduce production of ApoB-100, thereby reducing levels of LDL-cholesterol in said subject.

7. The method of claim 6 wherein said compound is selected from bufadienolide compounds comprising a 2H-pyran-2-one-5-yl moiety and cardenolide compounds comprising a 5H-furan-2-one-4-yl moiety.

8. The method of claim 7 wherein said compound is selected from digoxin, convallatoxin, proscillaridin, digitoxin, lanatoside C, ouabain, gitoxin, peruboside, strophanthidin and digoxigenin.

9. The method of claim 8 wherein said compound is selected from digitoxin and proscillaridin.

10. The method of claim 9 wherein said compound is provided is provided in a composition comprising a nanomolar concentration thereof.

11. The method of claim 6 wherein said mammalian subject is human.

12. A method of treating hypercholesterolemia, said method comprising administering to a human subject in need thereof a therapeutically effective amount of a compound of a formula 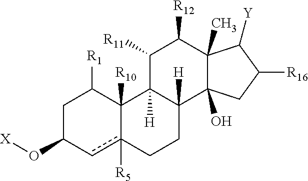
wherein X is selected from hydrogen (H), monosaccharide, disaccharide and polysaccharide moieties; Y is selected from 2H-pyran-2-one and 5H-furan-2(5H)-one moieties; R₁ is selected from H, hydroxyl (—OH), monosaccharide, disaccharide and polysaccharide moieties; R₁₀ is selected from H, methyl, hydroxymethyl, —OH and formyl (—(H)C═O) moieties; R₁₁ and R₁₂ are independently selected from H and —OH; R₁₆ is selected from H, —OH and acetate (—OC(═O)CH₃) moieties; and [symbol] represents either a single bond or a double bond, providing R₅ is selected from H, methyl and —OH moieties where said bond is a single bond, thereby reducing levels of LDL-cholesterol in said subject.

13. The method of claim 12 wherein said compound is selected from bufadienolide compounds comprising a 2H-pyran-2-one-5-yl moiety and cardenolide compounds comprising a 5H-furan-2-one-4-yl moiety.

14. The method of claim 13 wherein said compound is selected from digoxin, convallatoxin, proscillaridin, digitoxin, lanatoside C, ouabain, gitoxin, peruboside, strophanthidin and digoxigenin.

15. The method of claim 14 wherein said compound is selected from digitoxin and proscillaridin.

16. The method of claim 15 wherein said compound is provided is provided in a pharmaceutical composition comprising a nanomolar concentration thereof.

17. A method of treating hypercholesterolemia, said method comprising administering to a human subject in need thereof a therapeutically effective amount of a compound selected from digoxin, convallatoxin, proscillaridin, digitoxin, lanatoside C, ouabain, gitoxin, peruboside, strophanthidin and digoxigenin, thereby reducing LDL-cholesterol levels in said subject.

18. The method of claim 17 wherein said compound is selected from digitoxin and proscillaridin.

19. The method of claim 17 wherein said compound is incorporated into a pharmaceutical composition.

20. The method of claim 19 wherein said compound is provided in a nanomolar concentration.
